(19) United States
(12) Patent Application Publication
(10) Pub. No.: US 2022/0047459 A1
(43) Pub. Date: Feb. 17, 2022

(54) Medicine Dispenser and Applicator

(71) Applicant: Peter Lindner, New York, NY (US)

(72) Inventor: Peter Lindner, New York, NY (US)

(21) Appl. No.: 17/402,469

(22) Filed: Aug. 13, 2021

Related U.S. Application Data
(60) Provisional application No. 63/065,323, filed on Aug. 13, 2020.

(51) Int. Cl. A61J 7/00

(52) U.S. Cl. CPC A61J 7/0053

(57) ABSTRACT

The present invention relates to the field of medicament dispenser and applicator, and specifically, to a medicament dispenser and applicator having a reservoir and a plurality of individual pipette dispensers having the ability store, dispense and apply medicament to an area of treatment.

### RELATED APPLICATIONS

This application claims the benefit of U.S. Provisional Patent Application No. 63/065,323 filed Aug. 13, 2020, which is hereby incorporated by reference in its entirety.

### FIELD OF INVENTION

The present invention relates to an apparatus for receiving medicaments and dispensing the medicine in accordance with recommended and/or prescribed dosage. Specifically, the apparatus houses and dispenses topical medicaments at a predetermined dosage while minimizing unnecessary bodily contact.

### BACKGROUND OF THE INVENTION

Whether a medicament is over-the-counter (“OTC”) or prescribed, it is important to use the medicament only as directed. For example, the user should not use more (or less) than directed, and the medicament should only be applied at the point of treatment. In the case of minoxidil, a medicament used for treating hair loss, over-dosage and absorption by improper body parts other than the scalp, may affect the heart and blood vessels, as well as cause other unwanted effects. Improper dosage and/or contact can cause dizziness, fast/irregular heartbeat, fainting, and chest pain.

Despite potential risks, a manufacturer's recommended instructions for use may not be the ideal means for application of the medicament. Consider the general instructions for the application of Minoxidil® solution: (i) gently apply 1 milliliter (mL) of the product with a dropper over desired area, and (ii) massage where hair is thinning. First, droppers are not the easiest device to maneuver. The user may inadvertently incorporate air bubbles into the dropper, resulting in frustration since air bubbles interfere with proper quantification. Second, despite instructions to massage the treated area, it would be preferable to avoid unnecessary contact with such area to minimize absorption and/or displacement of the medicament by other parts of the body. Third, even if the medicament is used as directed, the medicament may be unnecessarily wasted due to inefficient application. For example, Minoxidil® may be difficult to control since it may flow in unwanted directions (e.g. on forehead and face). If the medicine is not caught in time, it is simply wasted, or worse, can cause hair to grow on face. Fourth, application of the medicament by hand can expose foreign agents such as bacteria and virus to the area of application.

Therefore, there is a need in the art for a dispenser capable for administering medicament in the proper dosage in a manner that facilitates application of the product without unnecessary bodily contact and waste. These and other features and advantages of the present invention will be explained and will become obvious to one skilled in the art through the summary of the invention that follows.

### SUMMARY OF THE INVENTION

Accordingly, it is an object of the present invention to provide a dispenser for medicament that accurately and uniformly administers over the point of treatment while providing a means for the user to safely distribute the medicament without the need for unnecessary bodily contact.

According to an embodiment of the present invention, this is accomplished by providing an apparatus comprising of a reservoir to hold the medicament flushed against a surface comprised of numerous pipette or nipple-like dispensers capable of dispensing a precise and accurate volume of a liquid medicament. The pipettes can be thought of as brush bristles.

According to embodiments, a liquid medicine dispenser and applicator comprising a reservoir configured to retain medicament, a fluid measurement and distribution system, and pipette dispensers configured to release said medicament.

According to embodiments of the current invention, the fluid measurement and distribution system is comprised of at least one sensor. Examples of applicable sensor include a point level measurement sensor and/or a capacitive level sensor. Alternate embodiments includes a combination of various sensors that can detect the liquid's volume, height, and/or weight within the reservoir.

According to embodiments of the current invention, the pipette dispensers are further comprised of mechanical release mechanism which may utilize controlled pistons to dispute liquid. Alternate embodiments utilize magnet and/or electromagnetic pumps to distribute the liquid within the pipette dispensers. Other pumps may be peristaltic pumps.

According to embodiments of the current invention, the liquid dispenser and applicator further comprise of a reservoir cap, which is further comprised of a reservoir cap opening. The reservoir cap opening enables an entry point for the medicament without the need to remove the reservoir cap. The reservoir cap also enables stable holding of the liquid receptacle so the liquid can be transferred without the constant need of the user to hold the receptacle and the current invention.

According to embodiments of the current invention, the liquid medicine dispenser further comprises of a pump chamber that enables the transfer of liquids such as medicament into the pipettes by means of a tube that extends from the reservoir into the pipette dispensers with intermediate stops along the way. The tube transverses the pipette dispenser plate from which the pipette dispensers extend.

According to an embodiment of the current invention, a method of distributing medicament comprising (i) adding liquid medicament to a liquid medicine dispenser and applicator comprising of a reservoir, a fluid measurement and distribution system, and pipette dispensers; (ii) setting a dispensing amount (which may be done in advance, electronically); and (iii) releasing the medicament in the pre-determined amount.

The foregoing summary of the present invention with the preferred embodiments should not be construed to limit the scope of the invention. It should be understood and obvious to one skilled in the art that the embodiments of the invention thus described may be further modified without departing from the spirit and scope of the invention.

### BRIEF DESCRIPTION OF THE DRAWINGS

Accompanying this written specification is a collection of drawings of exemplary embodiments of the present invention. One of ordinary skill in the art would appreciate that these are merely exemplary embodiments, and additional and alternative embodiments may exist and still be within the spirit of the invention as described herein.

FIG. 1 is a perspective view of an embodiment of the current invention.

FIG. 2 is an exploded view of an embodiment of the current invention.

FIG. 3 is another perspective view of an embodiment of the current invention illustrating some internal components.

FIG. 4 is a perspective view of a feature of the current invention.

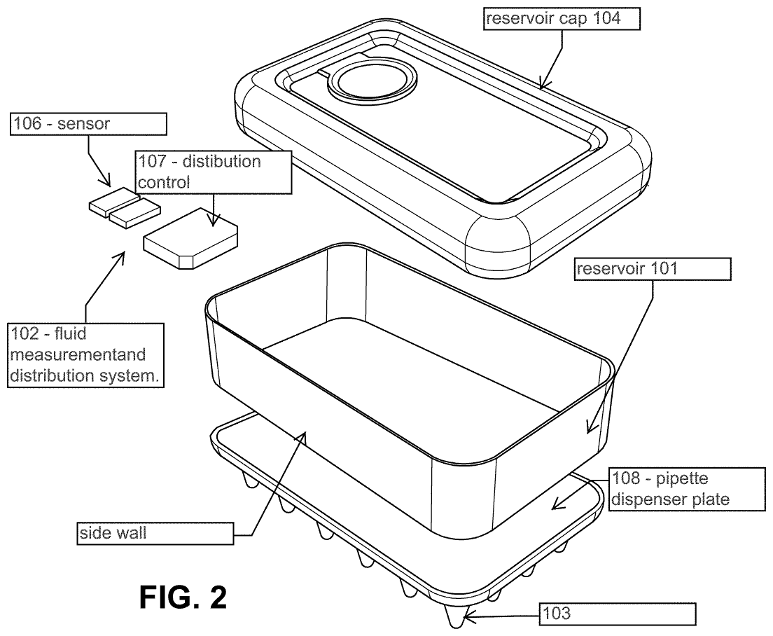

FIG. 2 illustrates potential top and bottom views. FIG. 2(A) illustrates the pipette dispensers as if they were configured in bristle form. FIG. 2(B) illustrates a potential hand grip affixed to a reservoir.

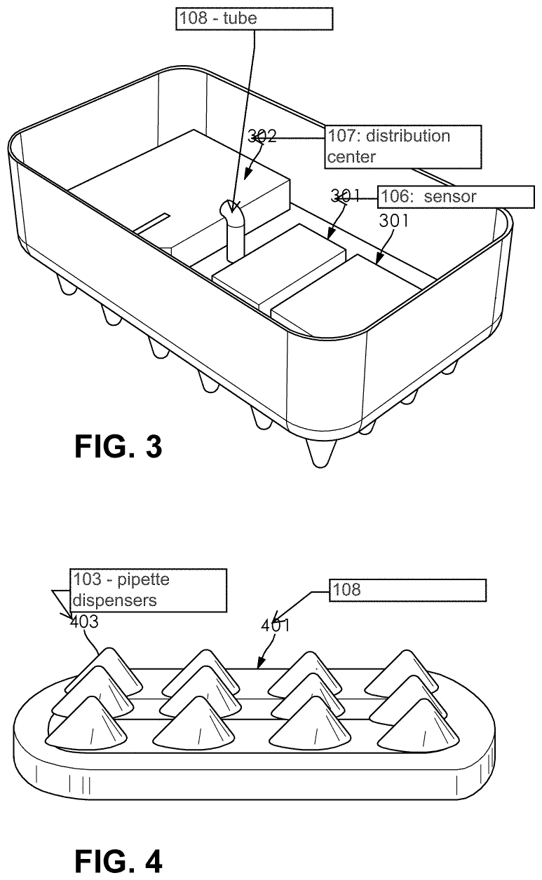

FIG. 3 is an exploded view of the fluid measurement and distribution system.

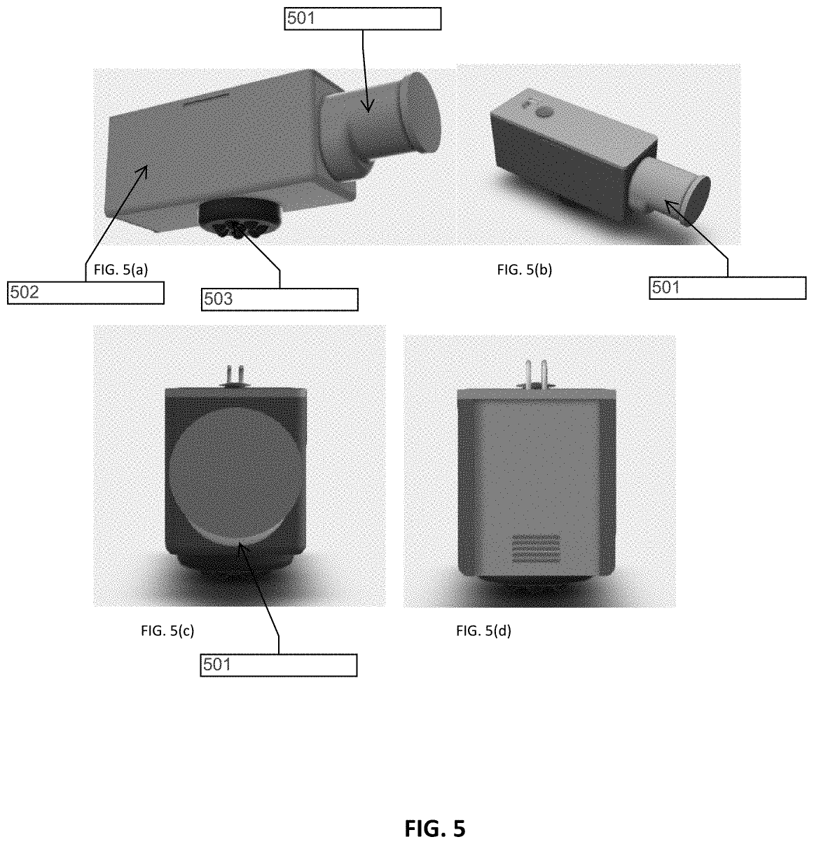

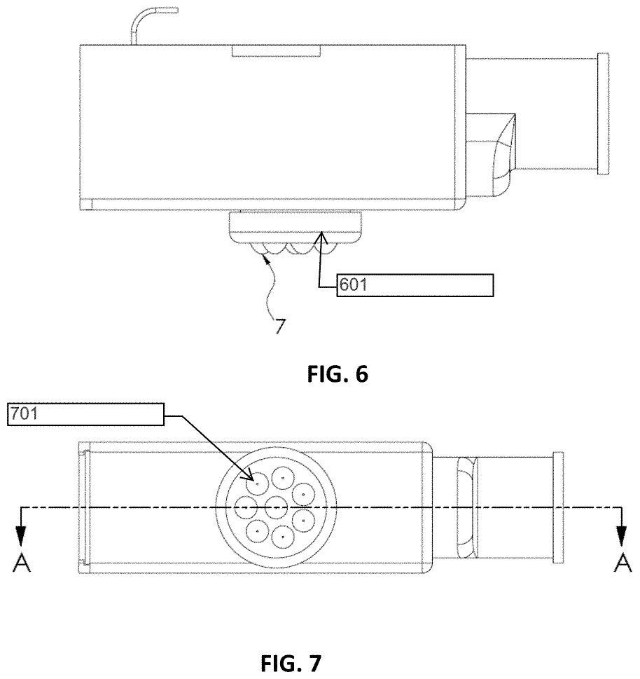

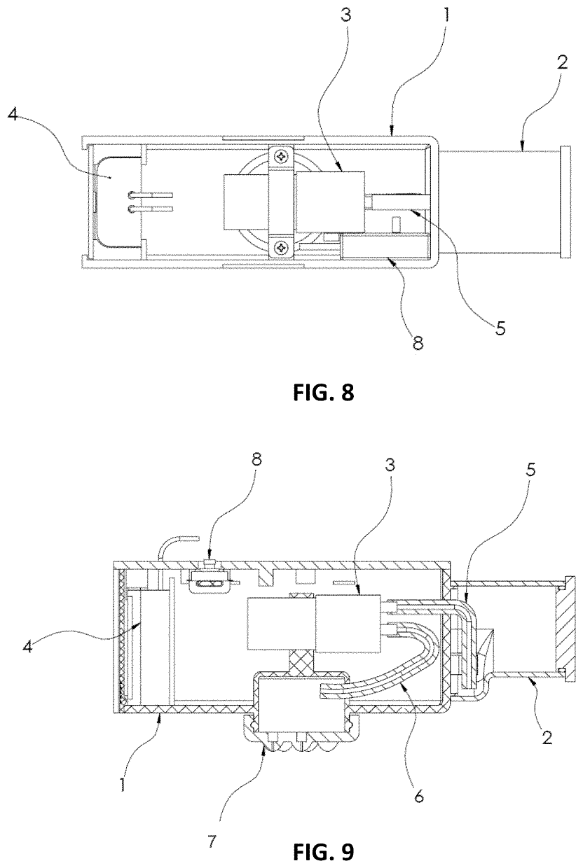

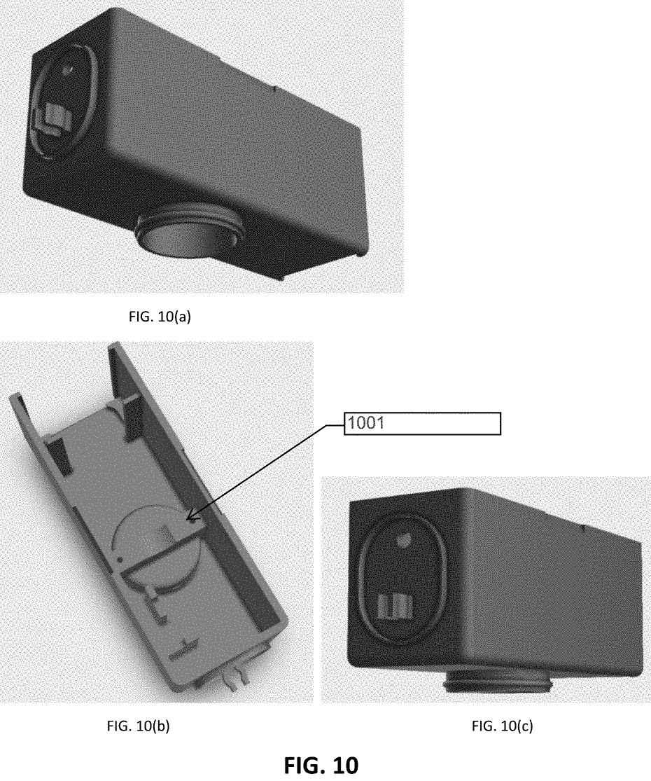

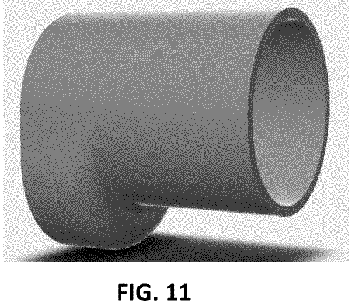

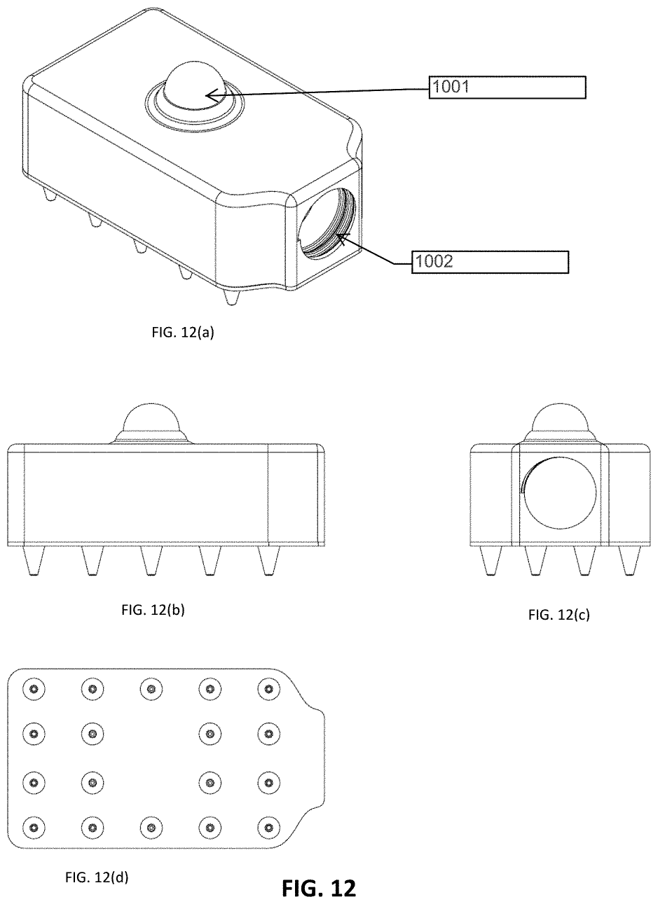

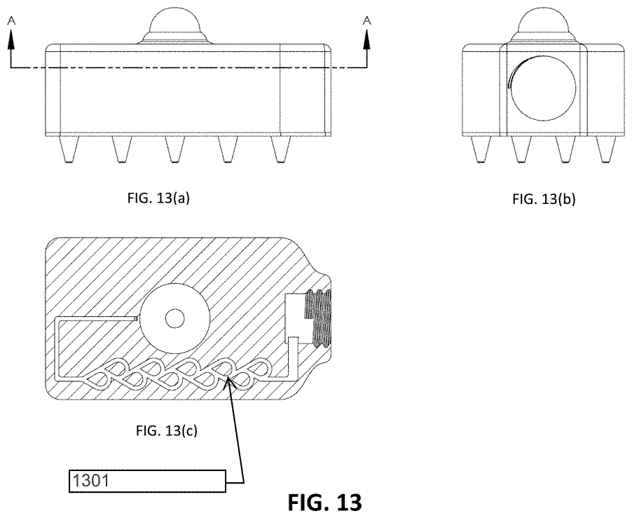

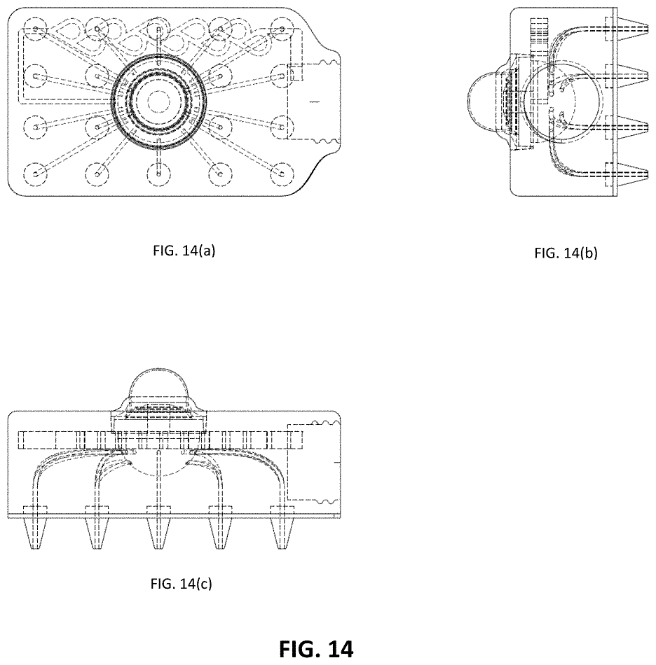

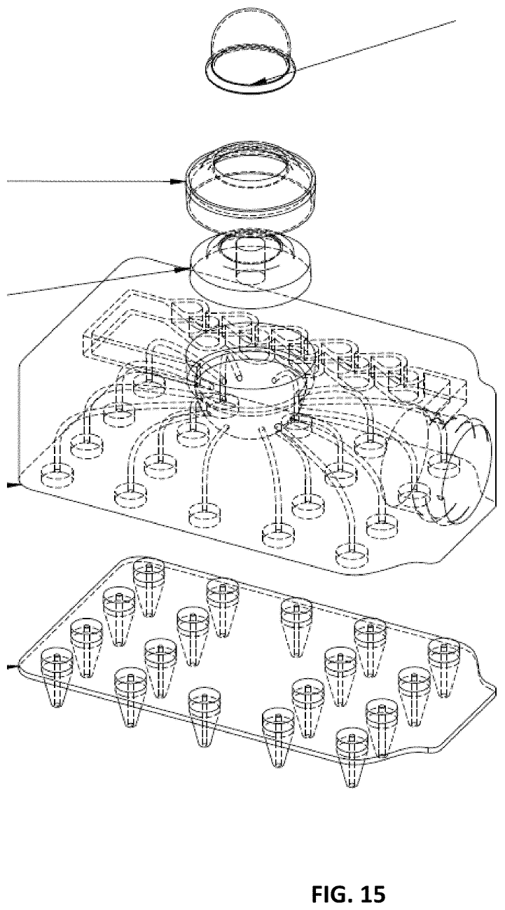

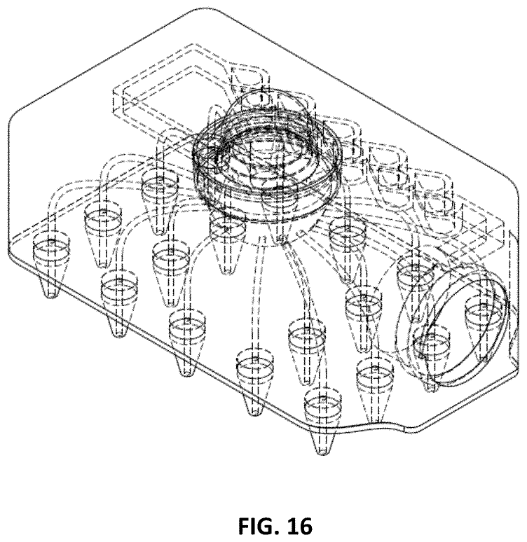

FIG. 4 is an illustration of bottom perspective view of a medicine dispenser and applicator in accordance with an embodiment of the present invention.

FIG. 5 are various views of an electronic embodiment of the current invention. FIG. 5(a) shows a front perspective view where the bottom surface is visible. FIG. 5(b) shows a front perspective where the top surface is visible. FIG. 5(c) shows a front view whereas FIG. 5(d) shows a rear view of an electronic embodiment of the current invention.

FIG. 6 shows a schematic view of an electronic embodiment of the current invention.

FIG. 7 shows a bottom view of an electronic embodiment of the current invention.

FIG. 8 shows a schematic view from a top perspective of the inner components of an electronic embodiment of the current invention.

FIG. 9 shows a schematic view from a side perspective of the inner comments of an electronic embodiment of the current invention.

FIG. 10 are various views of a main body housing of an electronic embodiment of the current invention. FIG. 10(a) shows a front perspective view where the bottom surface is visible. FIG. 10(b) shows a top view of a hollowed main body housing. FIG. 10(c) shows another front perspective view of an electronic embodiment of the current invention.

FIG. 11 shows a three dimensional rendering of the reservoir and fill port of an embodiment of the current invention. This reservoir and fill port may be used for both the electronic and mechanical embodiments.

FIG. 12 are various views of a manual or mechanical embodiment of the current invention. FIG. 12(a) shows a front perspective view where the top surface is visible. FIG. 12(b) shows a side view of a manual embodiment of the current invention. FIG. 12(c) shows a front view whereas FIG. 12(d) shows a bottom view of a mechanical embodiment of the current invention.

FIG. 13 are various schematic views of a manual embodiment of the current invention. FIG. 13(a) shows a side view of a manual embodiment of the current invention. FIG. 13(b) shows a front view of a manual embodiment of the current invention. FIG. 13(c) shows an internal view of a manual embodiment of the current invention.

FIG. 14 are internal schematic views of a manual embodiment of the current invention. FIG. 14(a) shows an internal view from a bottom perspective of a manual embodiment of the current invention. FIG. 14(b) shows a side view from a rear perspective of a manual embodiment of the current invention. FIG. 14(c) shows a rightside internal view of a manual embodiment of the current invention.

FIG. 15 is an exploded view of a manual embodiment of the current invention.

### DETAILED DESCRIPTION OF THE INVENTION

In the Summary above, the Detailed Description herein, the Claims below, and in the accompanying drawings, reference is made to particular features of the current invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Whenever a reference herein is made to a method comprising two or more defined steps, the defined steps can be carried out in any order or simultaneously (except where the context excludes that possibility), and the method can include one or more other steps which are carried out before any of the defined steps, between two of the defined steps, or after all of the defined steps (except where context excludes that possibility).

The present invention generally relates to a dispensing system and applicator (100) for topically administering liquid medicine. Specifically, embodiments of the invention relate to an apparatus comprising of the following components: (i) a reservoir that houses the medicine, (ii) a fluid measurement and distribution system, and (iii) pipette or cone-like dispensers that administer and topically distribute the medicine by avoiding unnecessary contact with body parts, specifically the hands, face and hair.

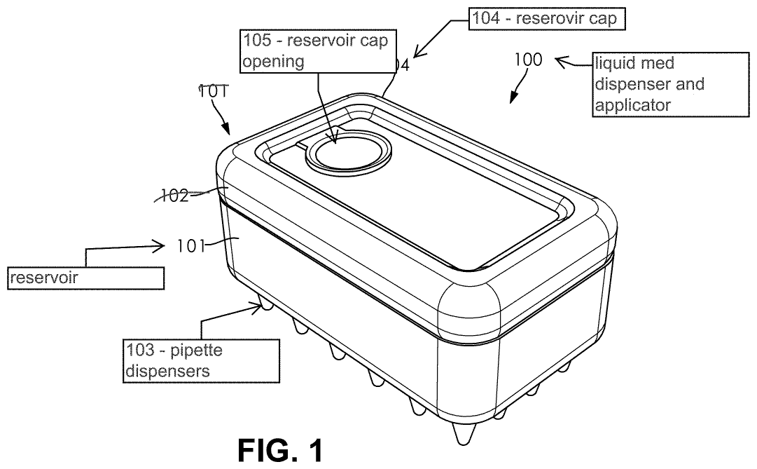

In the preferred embodiment, each component is planar and stacked, thereby comprising (from top to bottom or other permutations of locations) of a reservoir-layer (101), a fluid measurement and distribution layer (102), and a cone-like dispenser layer (103). See FIG. 1. Each layer is affixed to another layer via an attachment means comprising of any methodology of engineering fits such as clearance fits, location/transition fits, and interference fits. In the preferred embodiment, the attachment means affixing one layer to another layer comprise of snap fits and tabs. Alternate embodiments include circular attachments, via snaps or screw-ons. However, there are various attachments means for fitting one layer onto another layer available in the prior art and one or ordinary skill in the art would recognize that any of such attachment means could be adapted for use with the present invention without departing from the spirit and scope of the present invention.

In general, the reservoir is large enough to house multiple doses of the medicament to enable repeated applications without refill. As an illustration, the reservoir is configured to store two (2) ounces of a medicament, which, in the case of Minoxidil®, is generally sufficient for thirty (30) applications of one or two a day. In a preferred embodiment, the top end of the reservoir is planar or sculpted to enable the user to easily grab and hold the current invention. See FIG. 1.

Embodiments of the current invention contemplate various means to enable transfer of the medicament into the reservoir component of the invention. According to one embodiment of the current invention, the reservoir comprises of a screw-on or snap-on lid that is able to securely attach to the reservoir body that houses the medicament for a clean transfer. According to an alternative embodiment, the reservoir opening comprises of a collapsible funnel that can be expended upon transfer and collapsed and capped when not in use. According to other alternative embodiments, the reservoir comprises of a pop-on or a screw-on attachment for transferring the medicaments from the original container into the current invention. In another alternate embodiment, rather than transfer the medicine in the reservoir, the original container which holds the medicine is affixed to either the reservoir or the pipette dispenser. In another alternate embodiment, there is an adapter that can attach the original medicine holder to the reservoir or the pipette dispensers. The adapter can add groves or ridges to the original medicine dispenser that to which the reservoir or the pipette dispensers can screw on. However, there is no limitation to the configuration of the adaptor. In one embodiment, the invention is accompanied with multiple adapters so that there is at least one configuration that can reversibly affix the original medicine container to the current invention.

According to alternate embodiments of the current invention, the reservoir comprises of a bottle-like body with a threaded end. In such embodiment, internal grooves “or threads” of the reservoir twist into an opening to the quantitative layer. The opening is angled at 30-35 degrees to allow the “female” reservoir end to reversible attach to the opening.

According to embodiments of the current invention, the reservoir may be rectangular, round, pill-shaped, or oval. According to some embodiments of the current invention, the reservoir component comprises of transparent plastic. This feature thereby allows the user to gauge whether a refill of the medicament is warranted to ensure that a proper dosage is always administered. According to embodiments of the current invention, the reservoir may be comprised of inflexible, flexible or moldable/bendable material.

According to the preferred embodiment of the current invention, the invention comprises of a reservoir with a re-closable opening on the top end (104). In one embodiment, the reservoir comprises of a cap affixed to the reservoir via a hinge mechanism that flips open and close for refilling of the medicament. Some hinges may have time delays. In such embodiment, the cap reversibly fits securely within a hole in the reservoir. In an alternate embodiment, the reservoir comprises of a removable top reversibly affixed to the body of the reservoir via an attachment means comprising of an engineering fit. In as yet another embodiment, the reservoir comprises of a top comprising of two parts: one part is securely and irreversibly secured to the fluid measurement and distribution system whereas the other party is reversibly fitted to the fluid measure and distribution system so that it may be opened and closed. Each part is attached to each other via a hinge mechanism or snap-together tabs so the reversible part may be opened and closed without affecting the fit of the irreversible part. In alternate embodiment, the removable top slides away from the body of the reservoir, yet still remains attached. According to embodiments of the current invention, the opposite (bottom) end of the reservoir connects with the fluid measurement and distribution system and comprises of a membrane or alternately ball-point shaped membrane or ball point.

According to alternate embodiments of the current invention, the top end of the reservoir is comprised of a hand grip. An exemplary embodiment of the hand grip is provided in FIG. 2(B). In this exemplary embodiment, the hand grip secures the hand against the planar end, namely at the back of the brush at the opposite side of the bristles) for convenient use. Alternate embodiments include a pocket affixed to the planar top end to enable the hand or one or more fingers to slide thorough and provide an additional layer of protection against unnecessary bodily contact. In one embodiment, the pocket extends from the planar surface of the reservoir and is compromised of the same material as the current invention. In alternate embodiments, the pocket is comprised of fabric such as cotton terry cloth.

According to embodiments of the current invention, on the opposite side of the reservoir comprises of a fluid measurement and distribution system (102); FIG. 3. In some embodiments, the fluid measurement and distribution system control the reservoir and pipette dispensers. Specifically, in such embodiments, the system regulates the quantity of medicament that enters the pipette dispensers, holds the medicament until needed, and then releases the medicament upon command. As an illustration, the fluid measurement and distribution system comprises of an electro-mechanical system. However, numerous types of electro-mechanical systems to control measure and distribution are available in the prior art and one or ordinary skill in the art would recognize that any of such systems could be adapted for use with the present invention without departing from the spirit and scope of the present invention.

According to some embodiments of the current invention, the fluid measurement and distribution system are operable via a power supply. As an illustration, a power supply can be in the form of a battery (including but not limited to electrochemical batteries or wireless batteries or wireless chargers as in electric toothbrushes) (302), which enables portability of the current invention and immunity to power disruptions. As a second illustration, the power supply is a conventional voltage converter circuit connected to a 110 V AC source such as a household electrical wall socket or a USB port or a charging station such as for electric toothbrushes and for cellphones. Charging may also be accomplished by means of wireless charging, e.g. using inductive charging.

According to some embodiments of the current invention, the fluid measurement and distribution system comprise of a control unit, for example in the form of a microprocessor chip or circuit board (301). The control unit is programmed to turn the invention on/off, aspirate the proper amount from the reservoir into the pipette dispensers, and administer the appropriate dosage of medicament. The control unit can be set to dispense at various quantities of total volume per application. It can be controlled remotely via cellphone, encrypted to prevent tampering, and using non-contact methods such as Bluetooth or other such wireless connections. It can be controlled by a circuit delivery amount of time for the amount to flow in the circuit to/from the pump.

According to some embodiments of the current invention, the fluid measurement and distribution system incorporate a spray pump mechanism comprising of a pump chamber regulated by the control unit. The spray pump mechanism may be fully or partially configured within the reservoir. To illustrate this embodiment, a siphon tube may be attached to the back end of the pump chamber and be oriented towards the bottom of the reservoir to ensure that as much liquid as possible is dispensed from the reservoir in a vertical orientation of the pipette dispenser. It should be appreciated that any number of configurations may be utilized to define a pump chamber that is in fluid communication with a liquid reservoir. The unit may also be powered by a push or squeeze, as one would squeeze the bulb top of a medicine dropper.

According to one embodiment of the current invention, the pump chamber operates via a piezoelectric motor. When voltage from the power source is applied, the input converts into precisely defined increments controlling a shaft that regulates how much medicament is aspirated or dispensed. According to another embodiment of the current invention, the pump chamber operates via a peristaltic motor. In such embodiment the power source regulates the displacement of fluids through a pump mechanism.

In some embodiments of the current invention, the fluid measurement and distribution system comprises of an air pump. According to this system, the medicament is forced out using compressed air. As an illustration of an air pump mechanism, the pump is controlled by a motor, which is operable via a push button. The motor may be configured to be on top of or below the reservoir. In one embodiment, the motor is battery-operated.

The fluid measurement and distribution system regulate the flow of medicament into the pipette dispensers. In some embodiments, the pipette dispensers are configured to extend from a bottom surface of the reservoir such that the medicament is transferred directly from the reservoir into the pipette dispensers. As an illustration, the bottom end of the reservoir comprises of mechanized flaps that separate the medicament in the reservoir from each pipette dispenser. The flaps define an opening in their open position allowing the medicament to fill within the pipette dispensers, and seal in their closed position. Upon signal, the flaps simultaneously move, allowing the medicament from the reservoir automatically drain into the pipette dispensers at the appropriate amounts.

In one embodiment, the pipette dispensers are hollowed nipple-like cones, while other embodiments envision thinner bristle-like projections. In another embodiment, the pipettes are flexible so that they gently touch the scalp, unlike a regular hairbrush, and have some “play” in their contact allowing the pipettes to cover an area greater than the diameter of the end of the pipette.

According to some embodiments of the current invention, the pipette dispensers are hollowed until ready to use. A tube running from the reservoir to the pipette dispensers transfers the medicament while the fluid measurement and distribution system controls the flow of the medicament.

The shape and size of the pipette dispenses must account for various factors. Accordingly, multiple embodiments of the current invention are envisioned. One factor is the dispensing rate of the medicament. The hole size of each pipette impacts the flow rate of the medicament. Depending on the viscosity of the medicament, a faster or slower flow rate may be desired. As one illustration, if the medicament is very viscous, it may be preferable to have a slower flow rate so that no residual medicament remains within the pipette dispensers. On the other hand, if a medicament has very low viscosity, the user may still opt to have slower flow rate to allow for greater control over administration and distribution.

According to alternate embodiments of the current invention, the pipette dispensers comprise of slotted holes that are able to reversibly open. As an illustration, the pipette dispensers comprise of a ball bearing mechanism that moves (e.g. via rotation) as the apparatus moves across the treatment area, such as the scalp. In detail, the ball-point pen bearing mechanism may also be spring loaded so that the flow of the medicament is uni-directional in a forward flow.

In one illustrative embodiment of the current invention, the pipette dispensers are hollow and comprised of silicone material. In alternate embodiments, the pipette dispensers comprise of hollow bristles. In the preferred embodiments, the pipette dispensers are comprised of soft silicone cones that gently vibrate and are able to provide for a soft massage upon the treatment area. In such case, the medicament is applied more uniformly, and can be time and/or strength limited.

According to embodiments of the current invention, the pipette dispensers integrated with the fluid measurement and distribution system extend from a base plate (401). In some embodiments, the base may be flat, while in other embodiments, the base may comprise of flexible material, such as rubber to obtain a pliable and/or springy and curved configuration. However, non-rubber materials can also be utilized in case of rubber allergies. According to some embodiments, the base plate may be removable to facilitate rinsing and cleaning of the pipette dispensers. Other embodiments contemplate a modular disassembly of the pipette dispenser so that various components of the pipette dispensers may be separated and reunited for easier clean. In some embodiments, the invention is accompanied with a flexible and plastic membrane that can enable safe cleaning in the dishwasher. In advanced models, the electrical components are sealed to be waterproof to enable cleaning by dishwasher. In such case, the current invention is capable of being washed in an electrical dishwasher.

According to some embodiments of the current invention, the pipette dispensers may be augmented with optional features. One example is incorporating a massage unit that vibrates, allowing for better distribution of the medicament as well as soothing the treatment area. In some embodiments, the vibrations are controlled by an internal timer so that the vibrations automatically stop after a set time period.

According to some embodiments of the current invention, the pipette dispensers have their own release mechanism. In some embodiments, the release mechanism is completely mechanical while other embodiments include electro-mechanical release means. The release mechanism comprises of an actuator that expels the medicament upon command but automatically returns to a closed position and thus prevent undesired leakage or drippage.

Embodiments of the current invention include optional features. In some embodiments, various sensors are electrically connected to the control unit, such as a refill sensor, that can indicate if the total volume of the medicament is running low. Other features include a usage meter that tracks the number of doses that have been dispensed and a clock/time feature that tracks the days of month and time for each application. It can also signal such information to a person's cell phone for analysis of use patterns and tracking information.

As an illustration, one example of a regulator that may be used to control the administration of the medicament is a Burkert® Fluid Control System. This system allows for the distribution of fluids at microliter dosages. Another exemplary embodiment uses the Takasago® Chemical Pump. Both of these systems exhibit features ideal for the current invention: small-sized, lightweight, and slim. For a medicament like Minoxidil® for hair loss, the general dosage is 1 mL per treatment. Therefore, if there are twenty pipette dispensers, then each pipette dispenser needs to only release 50 uL of the medicament (since 50 microLiters times 20=1 milliLiter).

The present invention also includes a method of using a medicine dispenser and applicator. In the preferred embodiment, the method comprises of filling a reservoir of a medicine dispenser and applicator in an amount sufficient for multiple applications; setting the medicine dispenser and applicator to release a specific total volume of dose per application at the relevant surface (e.g. scalp); releasing the appropriate volume of total medicament through individual pipette dispensers; and massaging the medicament onto the surface until properly distributed. For general applications, the medicament is massaged until absorbed by the body. However, usage depends on the nature of the medicament as well as instructions provided by a physician or pharmacist.

According to certain embodiments, the current invention can be controlled remotely, for example by cell phone, to enable usage by non-contact methods. To illustrate, the current invention may be controlled by means of short-range communication technologies such as near-field communication and Bluetooth. Alternate means include using wireless technology to remotely control the current invention such as wireless fidelity (“wi-fi”) and radio frequency identification (RFID), which can be in “read-only” or “read-write” modes.

Embodiments of the current invention may be developed by plastic molding via 3D printing technology. The body (comprising of the reservoir and pipette dispensers) may be molded from relatively inexpensive plastic materials and may have any desired aesthetic shape. The 3D printing can make the entire device, since a multi-jet printer can have 2-3 different materials (from soft/flexible to hard).

Significantly, in the preferred embodiment all components of the current invention, other than electrical components, are machine washable, i.e. capable of being cleaned in an electronic dishwasher. In alternate embodiments, certain components are machine washable. As examples, each of the reservoir and pipette dispensers are washable. If a version of the current invention comprises of a removable cover, the cover may also be machine washable.

According to a second embodiment of the current invention, the quantity of medicine dispensed is controlled by a relay timer. Specifically, the relay timer is programmed to a particular timing sequence. This timing sequence can be adjusted according to a process defined by the manufacturer of the relay timer. The duration of timing sequence is proportional to the amount of medicament that is dispensed through the current invention.

The second embodiment is comprised of a reservoir and fill port connected to a main body housing. The main body housing holds the majority of the electronic components to operate the invention. Extending from the bottom surface of the main body housing is the bristle assembly.

In greater detail, the reservoir and fill port is a generally tubular unit that receives and houses the medicament. The bottom, rear section of the reservoir and fill port bulges out to create the reservoir cavity for storing the medicament for use. The front end of the reservoir and fill port is configured with a reservoir cap that can be reversibly removed. The opposite end of the reservoir and fill port is configured with a partition wall that separates the medicament in the reservoir from the inner components of the main body housing. An inlet tube, preferably in the shape of an L, has one end immersed in the reservoir and fill port while the opposite end is affixed to the pump within the main body. The inlet tube is configured to create a seal against the partition so that the medicament in the reservoir does not leak into the main body.

According to embodiments, the main body housing holds much of the electronic components of the current invention, including the button switch. The main body housing also holds the pump, the outlet tubing, the timer relay, the battery pack, and the bristle assembly. The inlet tube is the only component that is positioned in both components; one end of the inlet tube extends into the reservoir while on the other end affixes to the pump.

The pump can be centrally located within the main body housing. At least two tubes directing the flow of medicament are connected to the pump. The first main type of tube is the inlet tube. The inlet tube originates in the reservoir and is threaded through the partition to connect to the pump. The second main type of tubing is the outlet tubing where the medicament is released to the bristle assembly. The outlet tubing traverses through a wall of the dispensing chamber. In both cases, the threading of the silicone tubing through the partition or wall is sufficiently tight to produce a seal such that no liquid can leak between the two surfaces.

The bristle assembly is comprised of a dispensing chamber situated predominantly within the main body housing and a bristle cap configured on the externally facing portion of the dispensing chamber. The dispensing chamber receives the medicament from the outlet tubing and ejects the medicament through the bristle cap, which is configured with numerous pipettes.

Dispensing of the medicament is controlled by a timer relay configured within the main body housing. When the user pushes the start button on the device, the relay timer receives this signal. The relay timer is programmed to send electric power to the pump, according to a particular timing sequence. This timing sequence can be adjusted according to a process defined by the manufacturer of the relay timer. When the pump is powered by activation of the button, it will pull liquid from the reservoir through the inlet tube, pressurize it and then transfer it to the dispensing chamber through the second tube. The liquid will flow out the end of the second tube and into the dispensing chamber. Accordingly, the relay timer is programed to allow the pump to run for a set amount of time.

The movement of medicament from the invention to the user is due to applying pressure to the current invention. In detail, once the internal pressure chamber is high enough, the liquid enters a set of small passages contained in the exposed face of the bristle cap so the liquid will flow out of the device through these bristles. If the current invention is positioned with the tips of the bristles in contact with the user's scalp, the liquid will flow onto their scalp.

According to another alternate embodiment of the current invention, the invention is manually operated. The manual embodiment broadly comprises of a reservoir, pipette dispensers, and a bulb assembly. In the exemplary embodiment, the manual embodiment is configured to dispense a fixed amount of medicament determined by the fixed capacity of the bulb. Alternate embodiments of the manual version contemplate a main body that is adaptable to various sizes of the bulb, thereby allowing for the dispensing of various quantities of medicament.

In an exemplary manual embodiment, the main body housing contains a body opening configured to receive and hold the original medicament container. As an example, the body opening is configured with a screw/thread design as a cap thread so the original medicament container can be screwed onto the present manual embodiment. The body opening may be configured in any design that can integrate with the original medicament container.

According to the exemplary manual embodiment, a tube housed within the main body is inserted into the original medicament container to transfer the medicament into the present invention. The tube is extended into at least one unit of a valvular conduit (also known as a Tesla valve). In the present invention, the Tesla valve ensures that the flow occurs in one direction, namely from the container to the centrally located reservoir.

According to the exemplary manual embodiment, there is at least one valvular conduit. However, alternate manual embodiments include more than one in various orientations. As examples, there may be two Tesla valve unites, each one traversing along a long side of the main body; or in alternate configurations positioned on top of one another.

Once the medicament flows through the Tesla valve unit(s), the medicament is emptied into a reservoir. The medicament is dispensed from the reservoir by action of the bulb assembly. The bulb assembly is comprised of a bulb or bladder situated upon a bulb bracket. The bulb bracket is maintained in position by means of a bulb base. The bulb is comprised of rubber material or similar material that can serve as a vacuum source. When the bulb is depressed, it aspirates the medicament; when it is released, the medicament travels from the reservoir to the pipette dispensers within the bristle pad.

A notable feature of the manual embodiment is that there is no back flow (or reverse flow) of the medicament. There are two significant features that effectuate the one direction flow of the medicament. The first feature is the Tesla valve. The Tesla valve is comprised of enlargements, recesses, projections, baffles, or buckets that force fluid moving in one direction to loop back on itself at various points along its length. The second feature is the size of the tubes that transport the medicament from the reservoir to the pipette dispensers. The current manual embodiment is optimized for the dispensing of 1 mL of Minoxidil. Based on studies of the flow dynamics of Minoxidil, it has been determined that a tube diameter of 1.0 mm-1.25 mm prevents the back flow of the medicament. The reasons as to why this tube diameter range prevents backflow of specifically Minoxidil are currently being investigated.

According to another manual embodiment, the invention is configured with two reservoirs: one reservoir at the outset and the other reservoir under the bulb assembly. In this embodiment, the main body is configured with a reservoir and fill port that receive and maintain the medicament. A reservoir cap configured with an O-ring seals the medicament within the reservoir and fill cap. The reservoir and fill port is separated from the main body housing by a partition or a wall. A tube threaded through the partition enables the flow of medicament from the reservoir and fill port to the main body. As an example, the tube is configured as a siphon to enable the medicament to travel from the reservoir and fill port to the main body. The siphon is further supported by at least one Tesla valve unit which promotes the flow of the medicament in one directly, namely from the reservoir and fill port to the reservoir of the bulb assembly.

Turning now to the figures, FIG. 1 illustrates an exemplary embodiment of the current invention. The liquid medicine dispenser and applicator (100) comprise of a reservoir (101), a fluid measurement and distribution system (not shown), and pipette dispensers (103). In the preferred embodiments, the reservoir is covered by a reservoir cap (104) that is reversibly fitted onto the reservoir to form a top service. In the preferred embodiment, the cap is fitted with a reservoir cap opening (105) where medicament can be added to the reservoir without the need for removing the reservoir cap. In various embodiments, the cap is configured with grooves to enable medicament receptacles, such as bottles, to be reversibly affixed to the reservoir cap opening.

Turning now to FIG. 2 is an exploded view of an exemplary embodiment of the current invention. Illustrated are the reservoir cap (104), the reservoir (101), and pipette dispensers (103). Also illustrated are the internal components of the fluid measurement and distribution system (102). In greater detail, the fluid measurement and distribution system comprises of at least one sensor (106) that can be of any time that can measure liquid capacity, volume, or level within the reservoir. As examples, the sensor may be a point level measurement sensor or a capacitive level sensor. Also illustrated is a distribution control (107). The distribution control regulates the amount or volume of liquid dispensed through the pipette. According to certain embodiments of the current invention, at least a portion of a side wall is comprised of clear material such as columnar sight glass. This feature allows the user to visually assess the level of medicament housed in the reservoir.

The distribution control may work in various ways. As a first example, the distribution control monitors the flow rate of liquid through the pipette dispensers. As an illustration, assume that the user desires to distribute a total of 1 mL of medicament. The distribution control then distributes a total of 1 mL through the pipette dispensers and then automatically shuts off the dispenser to prevent further liquid from being distributed. As a second example, the distribution control aspirates a predetermined amount of liquid and then dispenses the entirety of the aspirated amount. As a third example, the distribution control calculates the weight, height, and/or volume of liquid. The correct distribution amount of medicament is accomplished by detecting or calculating the necessary changes in weight, height, or volume. Simply as an illustration, if the medicament height begins at 5 cM but a total of 1 mL of medicament needs to be distributed, the corresponding height that equates to 1 cM is the trigger that turns off the distribution. Various tools can be used to regulate the flow of liquid medicament such as a flow meter.

Also shown in FIG. 2 is a plate (108) from which the pipette dispensers extend. In the preferred embodiment, the plate comprises of a top surface that serves as a natural boundary between the reservoir and the pipette dispensers and serves as a stable framework from which the pipette extend.

Turning now to FIG. 3 is another perspective view illustrating the interior of the reservoir. Visible here are the liquid sensors (107) capable of determining liquid weight, height, or volume and the distribution center (107). Also included is the pump chamber tube which transfers the liquid from the reservoir to the pipette dispensers.

Turning now to FIG. 4 is an exemplary embodiment of the pipette dispensers. In this exemplary embodiment, the pipette dispensers are distributed equidistantly so that the medicament may also be distributed evenly upon the user. In alternate embodiments, the pipette dispensers are randomly distributed. Further, in the preferred embodiments, the pipette dispensers are conical in shape. However, alternate shapes are also envisioned. One alternate embodiment may be tube-shaped pipette dispensers. Another alternate embodiment may include pipette dispensers individually configured in spherical form.

Turning now to FIG. 5 are various views of an electronic version of the current invention. Visible here are the reservoir and fill port (501), main body housing (502), and bristle (or pipette) pad and bristles (503). The reservoir and fill port is covered by reservoir cap that is utilized to prevent spillage of medicament stored within the reservoir and fill port. Turning now to FIG. 6 is a schematic view of an electronic version of the current embodiment. Notable in FIG. 6(a) is the bristle pad comprising of pipettes. In this embodiment, the majority of the bristle pad is configured externally, outside of the main body housing. In alternate embodiments, the bristle pad may be more or less projected, and therefore more or less visible. In some embodiments, the entirety of the bristle pad is configured within the main body housing where only the bristles or pipettes are extended out of the main body housing for application of the medicament.

Turning now to FIG. 7 is a bottom view of an electronic version of the current invention. Notable here are the hollowed bristles the dispense medicament.

Turning now to FIG. 8 is a schematic of the internal components of an electronic embodiment. Visible here are the housing (1), reservoir and fill port (2), the pump (3), the battery pack (4), the inner tubing, the inlet housing (5) and timer replay (8).

Turning now to FIG. 9 is a schematic of the internal components of an electronic embodiment of the current invention. Notable here are the illustration of the inlet tube (5) and the outlet tube (6). One end of the inlet tube is positioned in the reservoir and fill port. It is important to note that the inlet tube does not reach the bottom surface of the reservoir and fill fort. If that were the case, activation of the invention would create pressure and rather than aspirate the medicament, there would develop a pressure seal which could hinder the uptake of medicament. The opposite side of the inlet tube is fitted through a partition between the reservoir and fill port and the main body housing and ultimately connected to the pump. The 90 degree configuration of the inlet port allows for the siphoning of the medicament into the pump. Meanwhile, the outlet port extends from the battery to the dispensing chamber (7). Pressing the button controlling the timer relay (8) activates instructions concerning how long the pump dispenses the medicament.

Turning now to FIG. 10 are various views of the main body housing for the electronic embodiment. Visible in FIG. 10(b) is the motor claim 1001).

FIG. 11 is a closer view of the reservoir and fill port.

Turning to FIG. 12 are various views of the mechanical/manual embodiment. Visible here is the bulb (or bladder) that is depressed for the activation of the current invention. Visible in FIG. 12(a) is a threaded opening capable of receiving the medicament in its original container.

Turning to FIG. 13 are various views of the mechanical/manual embodiment. Notable in FIG. 13(c) is a Tesla valve unit (1301) that extends from a tube within the reservoir and fill port.

Turning to FIGS. 14-16 are various views of the mechanical/manual embodiment. Illustrated in these pictures are some of the tubing involved in the transport and dispensing of the medicament.

Optional features include a hand grip feature which reinforces the handling of the current invention. Specifically the hand grip is an extension that may extend from the pipette plate (108) through which the user's hand slides and grips the current invention on its side. If the user loses its grip, the hand grip may aid in preventing the current invention from falling.

Another optional feature to be incorporated in any embodiment is a timer so as to prevent or minimize overdosing on the medicament.

Embodiments of the invention can be made of any material of any softness. Examples of some materials used in the construction of the present invention include plastic and wood.

It is understood that the above-described embodiments are illustrative of only a few of the many possible specific embodiments, which can represent applications of the invention. Numerous and varied other arrangements can be made by those skilled in the art without departing from the spirit and scope of the invention.

1. A liquid medicine dispenser and applicator comprising:
a reservoir configured to retain medicament,
a fluid measurement and distribution system; and
pipette dispensers configured to release said medicament.

2. The fluid measurement and distribution system of claim 1 further comprised of a sensor.

3. The sensor of claim 2 further defined as a point level measurement sensor.

4. The sensor of claim 2 further defined as a capacitive level sensor.

5. The pipette dispensers of claim 1 further comprised of a mechanical release mechanism.

6. The reservoir of claim 1 comprised of a removable reservoir cap.

7. A top surface of said reservoir cap of claim 5 further comprising of a reservoir cap opening.

8. The pipette dispensers of claim 1 configured upon a plate receiving medicament for dispensing.

9. A liquid medicine dispenser and applicator comprising:
a reservoir and fill port adjoined to a main body housing, wherein a partition separates said components,
a pump that regulates uptake and release of liquid,
and a bristle cap that dispenses liquid regulated by said pump,
wherein a quantity of liquid dispensed is determined by a set time duration programmed on a timer relay.

10. The liquid medicine dispenser of claim 9 further comprising of a dispensing chamber that receives liquid uptake from said reservoir and fill port.

11. A liquid medicine dispenser and applicator comprising:
a main body configured with at least one Tesla valve, a bulb assembly, and a bristle pad configured with a multitude of pipette dispensers.

12. The liquid medicine dispenser and applicator of claim 11 further comprised of a reservoir wherein said bulb assembly regulates flow of liquid into said reservoir.
